(19) United States
(12) Patent Application Publication
(10) Pub. No.: US 2011/0053230 A1
(43) Pub. Date: Mar. 3, 2011

(54) METHOD FOR CONTROLLING RATE OF LOWERING MOLECULAR WEIGHT OF POLYSACCHARIDES CONTAINED IN CELLULOSIC BIOMASS, AND METHOD FOR PRODUCING SUGAR, ALCOHOL, OR ORGANIC ACID

(75) Inventors: Kazuhide TABATA, Nishikamo-gun (JP); Haruo Takahashi, Ogaki-shi (JP); Hisashi Miyafuji, Kyoto-shi (JP)

(73) Assignee: TOYOTA JIDOSHA KABUSHIKI KAISHA, Toyota-shi (JP)

(21) Appl. No.: 12/707,083

(22) Filed: Feb. 17, 2010

(30) Foreign Application Priority Data
Aug. 25, 2009 (JP) 2009-194226

(51) Int. Cl. C13K 1/02; C12P 7/04; C12P 7/40

(52) U.S. Cl. 435/136; 127/37; 435/157

(57) ABSTRACT

An object of the present invention is to provide, with regard to a method for lowering the molecular weight of polysaccharides contained in a cellulosic biomass by mixing the cellulosic biomass with ionic liquid, a method for controlling such rate of lowering of molecular weight. Also, a method for producing sugar, alcohol, or organic acid using the controlling method is provided. The method comprises mixing a cellulosic biomass with ionic liquid under an atmosphere with a partial pressure ratio differing from that of air. Under such an atmosphere with oxygen partial pressure higher than that of air, the rate of lowering molecular weight can be increased, and under an atmosphere with nitrogen partial pressure or carbon dioxide partial pressure higher than that of air or a reduced-pressure atmosphere, the rate of lowering molecular weight can be decreased.

### BACKGROUND OF THE INVENTION

1. Field of the Invention

With regard to a method for lowering the molecular weight of polysaccharides contained in a cellulosic biomass by mixing the cellulosic biomass with ionic liquid, the present invention relates to a method for controlling the rate of such lowering of molecular weight. Furthermore, the present invention relates to a method for producing sugar, alcohol, or organic acid using such controlling method.

2. Background Art

A cellulosic biomass is mainly composed of cellulose, hemicellulose, and lignin. In particular, cellulose or hemicellulose that is a polymeric form of glucose or xylose is a regenerable carbohydrate resource. Alcohol or organic acid represented by ethanol, lactic acid, or the like can be produced as a raw material with the use thereof. Hence, such cellulose or hemicellulose has received attentions as an alternative to petroleum as an energy source.

To produce alcohol or organic acid from a cellulosic biomass, cellulose or hemicellulose contained in a cellulosic biomass is hydrolyzed (glycosylated) to constitutive monosaccharides. Monosaccharides are then converted to alcohol or organic acid via fermentation. As a method for hydrolyzing (glycosylating) cellulose or hemicellulose to constitutive monosaccharides, a method using ionic liquid as described in JP Patent Publication (Kohyo) No. 2005-506401 A or No. 2009-79220 A is currently receiving attention. Such method is applied to a cellulosic biomass, so that the molecular weight of polysaccharides such as cellulose and hemicellulose contained in such cellulosic biomass can be lowered.

### SUMMARY OF THE INVENTION

Despite of the above, regarding a technique for treating a cellulosic biomass with ionic liquid, no method for controlling the rate of lowering the molecular weight of cellulose and/or hemicellulose has previously been known. In view of the above described circumstances, an object of the present invention is to provide, regarding a method for lowering the molecular weight of polysaccharides contained in a cellulosic biomass by mixing the cellulosic biomass with ionic liquid, a method for controlling the rate of such lowering of molecular weight. Another object of the present invention is to provide a method for producing sugar, alcohol, or organic acid with the use of the controlling method.

As a result of intensive studies to achieve the above objects, the present inventors have discovered that the rate of lowering the molecular weight of polysaccharides contained in a cellulosic biomass can be varied by changing the atmosphere in which the cellulosic biomass and ionic liquid are mixed. Thus the present inventors have completed the present invention.

The present invention encompasses the following.

The method for controlling the rate of lowering the molecular weight of polysaccharides contained in a cellulosic biomass according to the present invention comprises mixing the cellulosic biomass with ionic liquid under an atmosphere with a partial pressure ratio differing from that of air.

In the method for controlling the rate of lowering the molecular weight of polysaccharides contained in a cellulosic biomass according to the present invention, the atmosphere preferably has oxygen partial pressure higher or lower than that of air. Also, the atmosphere preferably has nitrogen partial pressure or carbon dioxide partial pressure higher than that of air. The atmosphere is preferably of a pressure lower than atmospheric pressure. The atmosphere also preferably has low oxygen partial pressure but has high nitrogen partial pressure or high carbon dioxide partial pressure and is in a reduced-pressure state.

Furthermore, in the method for controlling the rate of lowering the molecular weight of polysaccharides contained in a cellulosic biomass according to the present invention, the ionic liquid is preferably a 1-ethyl-3-methylimidazolium salt, and it is more preferably 1-ethyl-3-methylimidazolium chloride.

In addition, the method for producing sugar according to the present invention comprises a pretreatment step of mixing a cellulosic biomass with ionic liquid under an atmosphere with oxygen partial pressure lower than that of air, a solid-liquid separation step of separating solid components from liquid components (both of which are obtained in the pretreatment step), and a glycosylation step of glycosylating by enzyme treatment the solid components separated in the above solid-liquid separation step. Here, the above atmosphere with nitrogen partial pressure higher than that of air is further preferable.

Also, the method for producing alcohol or organic acid according to the present invention comprises a pretreatment step of mixing a cellulosic biomass with ionic liquid under an atmosphere with oxygen partial pressure lower than that of air, a solid-liquid separation step of separating solid components from liquid components (both of which are obtained in the above pretreatment step), a glycosylation step of glycosylating by enzyme treatment the solid components separated in the above solid-liquid separation step, and a fermentation step of fermenting a sugar component obtained in the above glycosylation step. Here, the above atmosphere further preferably has nitrogen partial pressure higher than that of air.

### EFFECTS OF THE INVENTION

Although the rate of lowering the molecular weight has been impossible to control when ionic liquid and a cellulosic biomass are mixed, the method for controlling the rate of lowering the molecular weight of polysaccharides contained in a cellulosic biomass according to the present invention makes it possible to simply control the rate of lowering the molecular weight of cellulose and/or hemicellulose.

### BRIEF DESCRIPTION OF THE DRAWINGS

FIG. 1 shows the results of HPLC analysis for soluble components at 3 hours after the initiation of treatment by which a cellulosic biomass and ionic liquid are mixed under various atmospheres.

FIG. 2 shows the results of HPLC analysis for soluble components at 24 hours after the initiation of treatment by which a cellulosic biomass and ionic liquid are mixed under various atmospheres.

FIG. 3 shows the results of GPC analysis for soluble components at 24 hours after the initiation of treatment by which cellulosic biomass and ionic liquid are mixed under various atmospheres.

FIG. 4 shows the results of X ray analysis for insoluble components at 3 hours after the initiation of treatment by which a cellulosic biomass and ionic liquid are mixed under various atmospheres.

FIG. 5 shows a graph of comparing the amounts of (synthesized) low-molecular-weight components of hemicellulose and cellulose contained in insoluble components at 24 hours after the initiation of treatment by which the cellulosic biomass and ionic liquid are mixed under various atmospheres.

### DETAILED DESCRIPTION OF THE PREFERRED EMBODIMENTS

Hereafter, the method for controlling the rate of lowering the molecular weight of polysaccharides contained in a cellulosic biomass and the method for producing sugar, alcohol, or organic acid according to the present invention are described in detail as follows.

The term “cellulosic biomass” in the present invention refers to a biomass comprising a complex of the crystal structure of cellulose fiber, and hemicellulose and lignin. In particular, the crystal structure of cellulose fiber and hemicellulose are treated as polysaccharides contained in a cellulosic biomass. Examples of the cellulosic biomass include waste materials such as lumber from thinning, construction and demolition waste, industrial waste, domestic waste, agricultural waste, waste lumber, materials remaining in the forested land, and waste paper. Also, examples of the cellulosic biomass include corrugated cardboard, waste paper, old newspapers, magazines, pulp, and pulp sludge. Furthermore, examples of the cellulosic biomass include pellets produced by pulverizing, compressing, and shaping waste lumber such as sawdust and wood shavings, materials remaining in the forested land, waste paper, or the like. Such cellulosic biomass may be used in any shape, but is preferably used after refinement, since refinement causes ionic liquid to easily act and accelerate the lowering the molecular weight of polysaccharides contained in a cellulosic biomass.

In the present invention, ionic liquid is mixed with a cellulosic biomass for lowering the molecular weight of the cellulosic biomass. At this time, examples of ionic liquid that can be used herein and that is applicable for lowering the molecular weight of a cellulosic biomass include, but are not particularly limited to, imidazolium-based ionic liquid, pyridine-based ionic liquid, alicyclic amine-based ionic liquid, and aliphatic amine-based ionic liquid. A compound to be used as such ionic liquid can be appropriately selected in view of the degree of lowering the molecular weight of cellulose and/or hemicellulose contained in a cellulosic biomass. In view of the degree of lowering the molecular weight of cellulose and/or hemicellulose, imidazolium-based ionic liquid composed of an imidazolium compound is preferably used. In particular, as an imidazolium compound, a 1,3-dialkylimidazolium salt is more preferably used. Among 1,3-dialkylimidazolium salts, 1-ethyl-3-methylimidazolium chloride is the most preferable for use.

In addition, examples of an imidazolium compound include a 1,3-dialkylimidazolium salt and a 1,2,3-trialkylimidazolium salt. Specific examples of a 1,3-dialkylimidazolium salt include 1-ethyl-3-methylimidazolium bromide, 1-ethyl-3-methylimidazolium chloride, 1-ethyl-3-methylimidazolium(L)-lactate, 1-ethyl-3-methylimidazolium hexafluorophosphate, 1-ethyl-3-methylimidazolium tetrafluoroborate, 1-butyl-3-methylimidazolium chloride, 1-butyl-3-methylimidazolium hexafluorophosphate, 1-butyl-3-methylimidazolium tetrafluoroborate, 1-butyl-3-methylimidazolium trifluoromethanesulfonate, 1-butyl-3-methylimidazolium(L)-lactate, 1-hexyl-3-methylimidazolium bromide, 1-hexyl-3-methylimidazolium chloride, 1-hexyl-3-methylimidazolium hexafluorophosphate, 1-hexyl-3-methylimidazolium tetrafluoroborate, 1-hexyl-3-methylimidazolium trifluoromethanesulfonate, 1-octyl-3-methylimidazolium chloride, 1-octyl-3-methylimidazolium hexafluorophosphate, 1-decyl-3-methylimidazolium chloride, 1-dodecyl-3-methylimidazolium chloride, 1-tetradecyl-3-methylimidazolium chloride, 1-hexadecyl-3-methylimidazolium chloride, and 1-octadecyl-3-methylimidazolium chloride. Examples of a 1,2,3-trialkylimidazolium salt include 1-ethyl-2,3-dimethylimidazolium bromide, 1-ethyl-2,3-dimethylimidazolium chloride, 1-butyl-2,3-dimethylimidazolium bromide, 1-butyl-2,3-dimethylimidazolium chloride, 1-butyl-2,3-dimethylimidazolium tetrafluoroborate, 1-butyl-2,3-dimethylimidazolium trifluoromethanesulfonate, 1-hexyl-2,3-dimethylimidazolium bromide, 1-hexyl-2,3-dimethylimidazolium chloride, 1-hexyl-2,3-dimethylimidazolium tetrafluoroborate, and 1-hexyl-2,3-dimethylimidazolium trifluoromethanesulfonate.

Furthermore, examples of pyridinium-based ionic liquid include an ethyl pyridinium salt, a butyl pyridinium salt, and a hexyl pyridinium salt. Specific examples of an ethyl pyridinium salt include 1-ethyl pyridinium bromide and 1-ethyl pyridinium chloride. Examples of butyl pyridinium salts include 1-butyl pyridinium bromide, 1-butyl pyridinium chloride, 1-butyl pyridinium hexafluorophosphate, 1-butyl pyridinium tetrafluoroborate, and 1-butyl pyridinium trifluoromethanesulfonate. Examples of a hexyl pyridinium salt include 1-hexyl pyridinium bromide, 1-hexyl pyridinium chloride, 1-hexyl pyridinium hexafluorophosphate, 1-hexyl pyridinium tetrafluoroborate, and 1-hexyl pyridinium trifluoromethanesulfonate.

Furthermore, examples of alicyclic amine-based ionic liquid include N,N,N-trimethyl-N-propylammonium bis(trifluoromethanesulfonyl)imide, N-methyl-N-propylpiperidinium bis(trifluoromethanesulfonyl)imide, N,N-diethyl-N-methyl-N-(2-methoxyethyl)ammonium bis(trifluoromethanesulfonyl)imide, and N,N-diethyl-N-methyl-N-(2-methoxyethyl)ammonium tetrafluoroborate.

Also, as described above, in imidazolium-based ionic liquid, pyridine-based ionic liquid, alicyclic amine-based ionic liquid, and aliphatic amine-based ionic liquid, anions may be either inorganic anions or organic anions. Examples of inorganic anions include Cl⁻, Br⁻, I⁻, NO₃⁻, BF₄⁻, PF₆⁻, and AlCl₄⁻. Also, examples of organic anions include CH₃SO₃⁻, CH₃CH(OH)COO⁻, lactic acid ions, CH₃COO⁻, CH₃OSO₃⁻, CF₃⁻SO₃⁻, (CF₃SO₃)₂N⁻, and (C₂F₅SO₂)₂N⁻. In particular, it is preferable to use ionic liquid containing Cl⁻ or ionic liquid containing CH₃COO⁻ as an anion. This is because in the case of such ionic liquid containing Cl⁻ or ionic liquid containing CH₃COO⁻, the dissolution rate of cellulose and/or hemicellulose contained in a cellulosic biomass is very high.

The method for controlling the rate of lowering the molecular weight of polysaccharides contained in a cellulosic biomass according to the present invention is characterized in that a cellulosic biomass and ionic liquid are mixed in an atmosphere with a partial pressure ratio differing from that of air.

Here, the term “partial pressure ratio differing from that of air” is, in other words, synonymous with the term “composition ratio (molar ratio) differing from that of air” of air comprising nitrogen, oxygen, carbon dioxide, and a trace gas such as argon.

Furthermore, the rate of lowering the molecular weight of polysaccharides can be calculated as an amount of soluble components (e.g, oligosaccharides and monosaccharides composing the polysaccharides) generated per unit of time. When a cellulosic biomass is mixed with ionic liquid, crystalline cellulose contained in the biomass is amorphized, molecular chains of amorphous (amorphized) cellulose and hemicellulose are gradually cleaved, so as to lower the molecular weight thereof to oligosaccharides and monosaccharides that are soluble in ionic liquid. Moreover, if the oligosaccharides and monosaccharides are left to stand in ionic liquid, such reaction of lowering the molecular weight further proceeds to finally yield excessively degraded products such as 5-HMF, furfural, and the like that inhibit a fermentation.

When oxygen partial pressure is higher than that of air, the rate of lowering the molecular weight of polysaccharides contained in a cellulosic biomass is increased. When the rate of lowering the molecular weight is increased, the molecular weight of cellulose and hemicellulose contained in the cellulosic biomass can be lowered within a shorter period of time. Therefore, a short period of time can be set for mixing ionic liquid with a cellulosic biomass. Accordingly, operating efficiency for lowering the molecular weight of all cellulose and hemicellulose to result in constitutive monosaccharides can be elevated. Also, if fermentation in ionic liquid is made possible, the method can be a low-cost and highly efficient method for producing alcohol or organic acid.

When oxygen partial pressure is set at a level higher than that of air, for example, oxygen partial pressure (oxygen volume ratio or molar ratio) preferably ranges from 30% to 100% and most preferably ranges from 70% to 100%. When the oxygen partial pressure is lower than 30%, the rate of lowering the molecular weight may not be significantly improved.

On the other hand, when oxygen partial pressure is lower than that of air, the rate of lowering the molecular weight of polysaccharides contained in a cellulosic biomass is decreased. Also, when nitrogen partial pressure or carbon dioxide partial pressure is higher than that of air or the atmosphere is a reduced-pressure atmosphere compared with atmospheric pressure, the rate of lowering the molecular weight of polysaccharides is decreased. When the rate of lowering the molecular weight is decreased, low-molecular-weight components of cellulose and hemicellulose can be easily maintained in solid form with molecular weights that do not allow their dissolution in ionic liquid. That is, a condition wherein cellulose and hemicellulose are not converted into soluble components such as oligosaccharides and monosaccharides can be maintained. Low molecular components of cellulose and hemicellulose, which are maintained in their solid form can be separated in such solid form from ionic liquid by solid-liquid separation. Specifically, as a result of solid-liquid separation, ionic liquid is recovered as a liquid component and lower-molecular-weight components of cellulose and hemicellulose are recovered as solid components. The thus recovered ionic liquid contains no soluble components from cellulose and hemicellulose. Therefore, when the ionic liquid is directly reutilized, excessively degraded components of cellulose and hemicellulose will never be formed. Also, the lowered molecular weight of cellulose and hemicellulose contained in solid components enables hydrolase such as cellulase to easily act. When sugar is produced by subjecting a cellulosic biomass to enzyme treatment, glycosylation efficiency is elevated. If alcohol or organic acid is produced from the sugar generated with such elevated glycosylation efficiency, production efficiency of alcohol or organic acid is also elevated.

When oxygen partial pressure is set at a level lower than that of air, for example, oxygen partial pressure (oxygen volume ratio or molar ratio) preferably ranges from 0% to 10% and most preferably ranges from 0% to 3%. When the oxygen partial pressure is higher than 10%, the rate of lowering the molecular weight may not be significantly decreased.

Also, when nitrogen partial pressure is set at a level higher than that of air, for example, nitrogen partial pressure (oxygen volume ratio or molar ratio) preferably ranges from 80% to 100% and most preferably ranges from 95% to 100%. When the nitrogen partial pressure is lower than 80%, the rate of lowering the molecular weight may not be significantly decreased. Similarly, when carbon dioxide partial pressure is set at a level higher than that of air, for example, carbon dioxide partial pressure (oxygen volume ratio or molar ratio) preferably ranges from 5% to 100% and most preferably ranges from 90% to 100%. If the carbon dioxide partial pressure is lower than 5%, the rate of lowering the molecular weight may not be significantly decreased.

To provide a reduced-pressure atmosphere compared with atmospheric pressure, for example, air pressure preferably ranges from 0.01 atmosphere to 0.8 atmosphere and most preferably ranges from 0.01 atmosphere to 0.5 atmosphere. If air pressure is higher than 0.8 atmosphere, the rate of lowering the molecular weight may not be significantly decreased.

In addition, a cellulosic biomass and ionic liquid are mixed by simply causing the cellulosic biomass to come into contact with ionic liquid. Alternatively, agitation, ultrasonic irradiation, vortexing, and the like may be carried out if necessary. Also, conventionally known methods such as filtration and centrifugation may be employed for solid-liquid separation.

Moreover, in the present invention, the temperature employed when a cellulosic biomass and ionic liquid are mixed to lower the molecular weight is not particularly limited, but preferably ranges from 60° C. to 150° C. and most preferably ranges from 80° C. to 120° C. to lower the molecular weight within a short period of time. A problem may arise such that the molecular weight cannot be sufficiently lowered by treatment at 60° C. or less. At the temperature of 150° C. or higher, a problem may arise such that the rate of lowering the molecular weight cannot be controlled.

The method for controlling the lowering of the molecular weight according to the present invention is applicable as a pretreatment step as described above, when monosaccharides and oligosaccharides (obtained by glycosylating polysaccharides contained in a cellulosic biomass via enzyme treatment) are produced. Cellulose is hydrolyzed via glycosylation by enzyme treatment to monosaccharides such as glucose. Hemicellulose is hydrolyzed to monosaccharides such as xylose, arabinose, and mannose. Examples of enzymes to be appropriately used for enzyme treatment include conventionally known enzymes capable of hydrolyzing cellulose and hemicellulose, such as cellulase and hemicellulase (xylase, arabinase, and mannanase). Also, a chemically synthesized enzyme, an enzyme prepared by purifying a product of a microorganism, or a microorganism capable of synthesizing an enzyme of interest may be mixed.

Also, alcohol or organic acid can also be produced by fermentation of the thus obtained sugar. As such alcohol, ethanol, propanol, butanol, glycerol, and the like can be produced. As such organic acid, lactic acid, acetic acid, citric acid, oxalic acid, succinic acid, β-hydroxybutyric acid, 3-hydroxypropionic acid, and the like can be produced.

Microorganisms to be used for the fermentation are not particularly limited, as long as they can produce a product of interest through the use of a sugar component obtained by a glycosylation step, such as monosaccharides and oligosaccharides. When ethanol is a product of interest, examples of such microorganisms include Saccharomyces cerevisiae and Schizosaccharomyces pombe. Also, when ethanol is a product of interest, bacteria such as Escherichia coli in which a gene group required for biosynthesis of ethanol using a monosaccharide or an oligosaccharide as a substrate has been introduced can also be used. Also, when lactic acid is a product of interest, conventionally known lactic acid-producing bacteria, such as bacteria belonging to the genus Lactobacillus can be exemplified. Also, Escherichia coli, yeast, or the like, in which a gene group required for biosynthesis of lactic acid using a monosaccharide or an oligosaccharide as a substrate, can be used.

The glycosylation step and the fermentation step according to the method for producing alcohol or organic acid of the present invention are carried out in separate vessels. At this time, the product is transferred to a fermentation vessel after completion of the glycosylation step and then the fermentation step may be carried out. A simultaneous glycosylation and fermentation step is also included herein, whereby glycosylation and fermentation are simultaneously carried out in the same vessel.

After completion of the fermentation step, a product of interest such as alcohol or organic acid can be recovered and generated by conventionally known techniques. For example, when ethanol is a product of interest, distillation, pervaporation membrane, and the like are applicable.

### EXAMPLES

The present invention will be described more specifically below in the following examples. The technical scope of the present invention, however, is not limited to the following Examples.

### Example 1

In this Example, an experiment for lowering the molecular weight of cellulose and hemicellulose contained in a cellulosic biomass was conducted under various atmospheres using 1-ethyl-3-methylimidazolium chloride as ionic liquid.

Specifically, 3 g of 1-ethyl-3-methylimidazolium chloride was added to a sealed round-bottom flask and then heated at 120° C. Under such conditions, 90 mg of absolutely dry western red cedar wood flour was added to the flask and then the mixture was agitated while supplying oxygen, nitrogen, carbon dioxide, pseudo-air, or each such gas containing moisture at a rate of 10 ml/minute. At 3 hours and 24 hours after initiation of the treatment, soluble components were separated from insoluble components by absorption and filtration. Similarly, 3 g of 1-ethyl-3-methylimidazolium chloride was added to a sealed round-bottom flask and then heated at 120° C. Under the conditions, 90 mg of absolutely dry western red cedar wood flour was added to the flask and then the mixture was agitated while reducing pressure using a pressure reducing pump to 0.01 atmosphere. At 3 hours and 24 hours after the initiation of the treatment, soluble components were separated from insoluble components by absorption and filtration.

Each soluble component (125 μl) was sampled and then mixed with 125 μl of dimethyl sulfoxide. The mixture was filtered and then the thus obtained filtrate was subjected to GPC analysis. Analytical conditions were as follows.

Sample inflow: 10 μl

Column: Shodex SB-803HQ

Eluant: dimethyl sulfoxide

Detector: refractive index detector and photodiode array

Column temperature: 60° C.

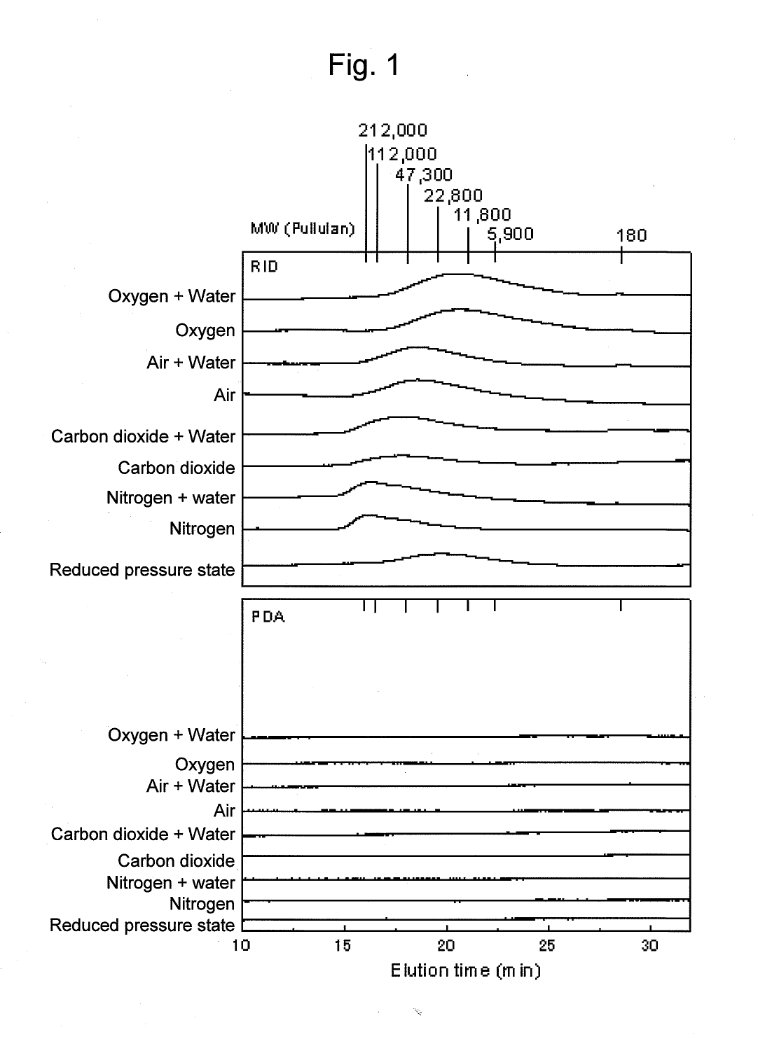

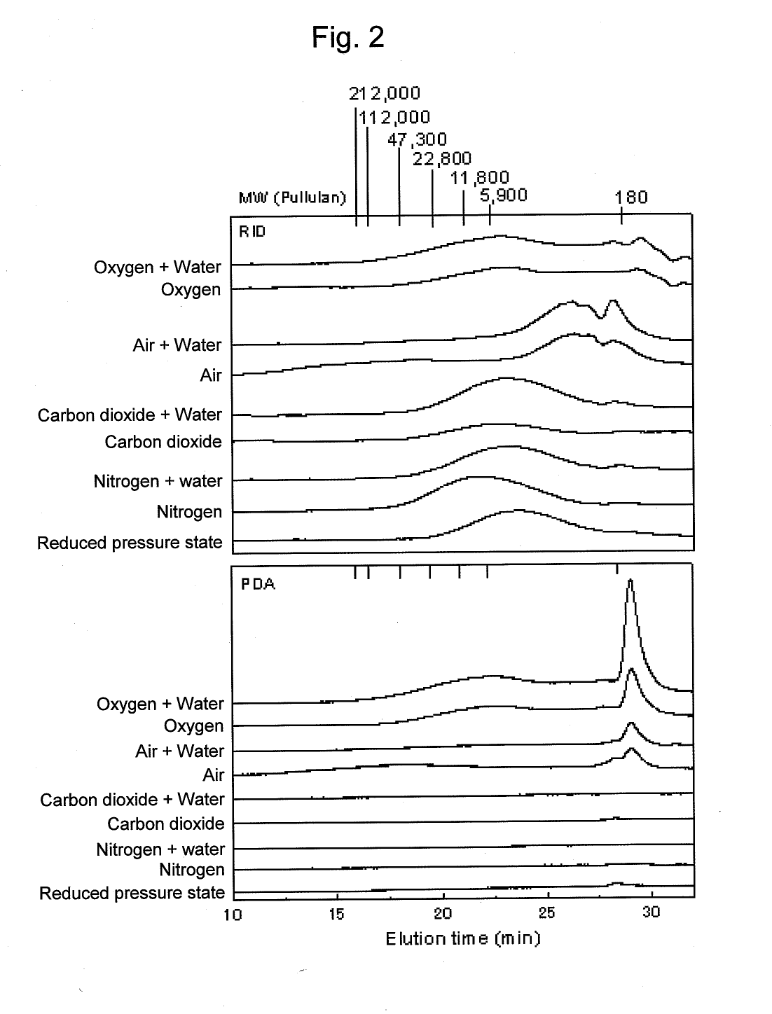

FIG. 1 shows the results at 3 hours after the initiation of treatment. FIG. 2 shows the results at 24 hours after the initiation of treatment.

After 3 hours, results of treatment under an oxygen or moisture-containing oxygen atmosphere showed peaks that had shifted further in the lower-molecular-weight direction, compared with the results for samples treated under the other atmospheres. Similar tendencies were also observed even after 24 hours.

Results of treatment under a carbon dioxide, moisture-containing carbon dioxide, nitrogen, moisture-containing nitrogen, or reduced-pressure atmosphere showed peaks that had shifted further in the higher-molecular-weight direction, compared with the results of treatment under a pseudo-air atmosphere. Specifically, it was understood that under an oxygen atmosphere, the rate of lowering the molecular weight of polysaccharides contained in a cellulosic biomass is increased; and under a carbon dioxide, nitrogen, or reduced-pressure atmosphere, the rate of lowering the molecular weight of polysaccharides contained in a cellulosic biomass is decreased. The result for each gas containing moisture did not significantly differ from those for the same gas containing no moisture. Therefore, it was shown that the presence or the absence of moisture does not significantly affect the rate of lowering molecular weight.

### Example 2

Each soluble component (10 μl) (separated in a manner similar to that in Example 1) was sampled and then mixed with 90 μl of distilled water. The solution was filtered and then the thus obtained filtrate was subjected to HPLC analysis. Analytical conditions were as follows.

Sample inflow: 10 μl

Column: Aminex HPX-87

Eluant: distilled water

Flow rate: 0.6 ml/min

Detector: refractive index detector and photodiode array

Column temperature: 85° C.

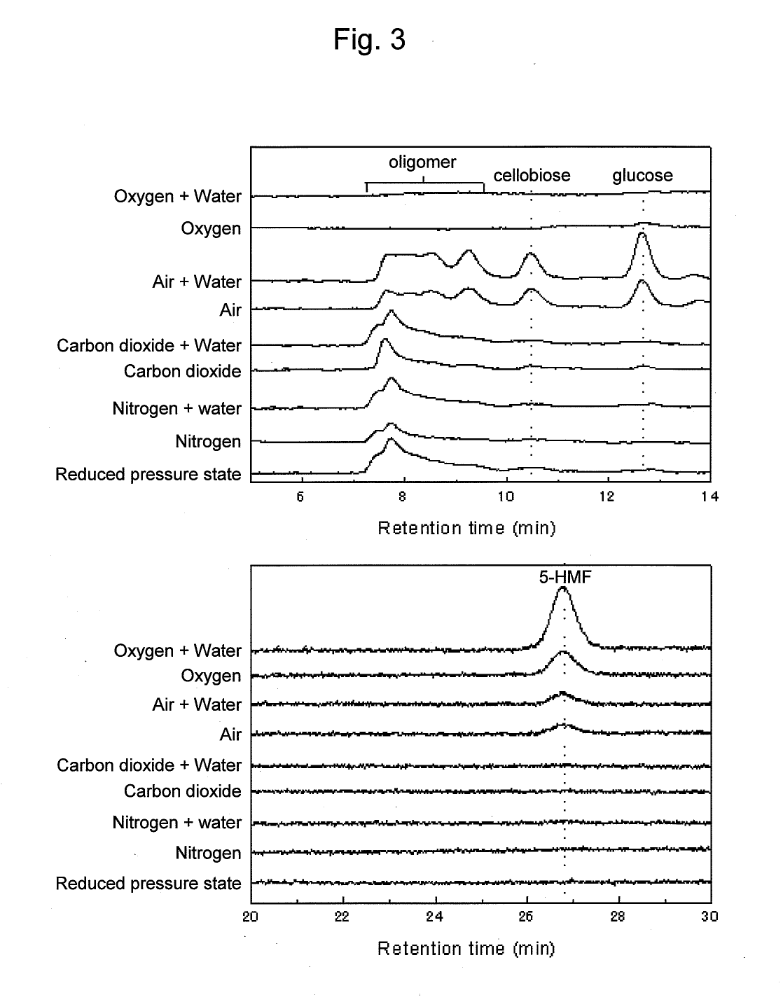

FIG. 3 shows the results at 24 hours after the initiation of treatment.

Results of treatment under a pseudo-air or moisture-containing pseudo-air atmosphere demonstrated that, glucose that is a monosaccharide of cellulose was contained at the highest level, and that oligomers and cellobioses of cellulose were also contained. 5-HMF that is an excessively degraded product wherein the molecular weight of glucose is somewhat further lowered was contained. On the other hand, results of treatment under an oxygen or moisture-containing oxygen atmosphere, oligomers, cellobioses, and glucose were not detected, and only 5-HMF the excessively degraded product was detected, suggesting a high rate of lowering molecular weight. Hence, when the molecular weight is lowered to a given level, the time for this treatment can be shorter under an oxygen atmosphere than that under an air atmosphere.

In the case of results of treatment under a carbon dioxide, moisture-containing carbon dioxide, nitrogen, moisture-containing nitrogen, or reduced-pressure atmosphere, even after 24 hours, only oligomers of cellulose were detected, and neither cellobioses nor glucoses were detected, suggesting a decreased rate of lowering molecular weight. Again, the results for each gas containing moisture did not significantly differ from those for the same gas containing no moisture. Therefore, it was shown that the presence or the absence of moisture does not significantly affect the rate of lowering molecular weight.

### Example 3

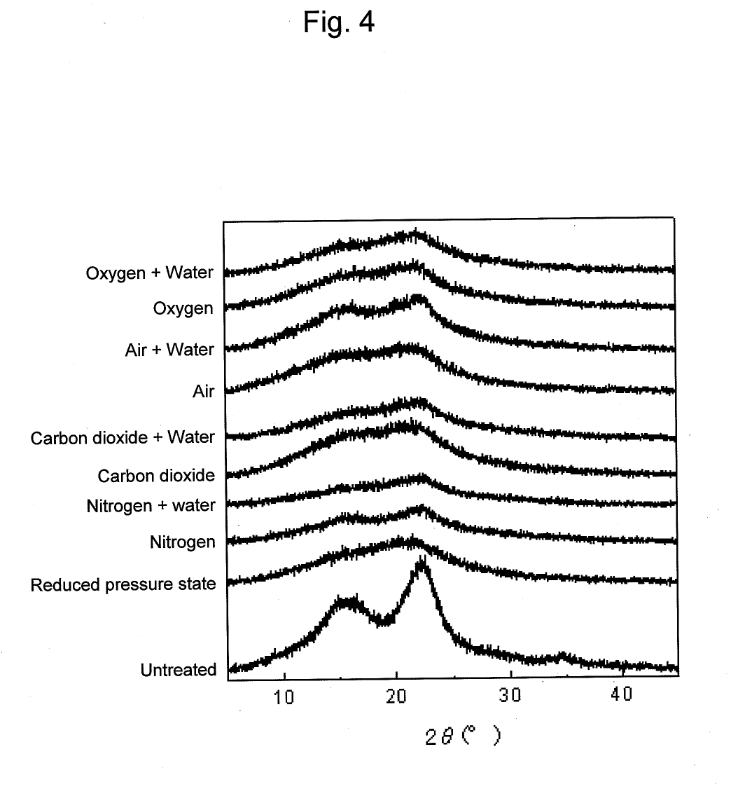

Each insoluble component (separated in a manner similar to that in Example 1) was thoroughly washed with dimethyl sulfoxide (DMSO) and then further washed with a sufficient volume of distilled water. The thus obtained residues were subjected to X-ray diffraction analysis using an X-ray diffractometer (Name of the apparatus: RINT2000 (manufactured by Rigaku Corporation)) under a voltage of 40 kv and a current of 30 mA. FIG. 4 shows the results of X-ray diffraction analysis at 3 hours after the initiation of treatment. Results when no treatment had been conducted showed peaks at position 2θ=22.6 indicating crystalline cellulose. However, the results regarding treatment with ionic liquid under any atmosphere showed no peaks at position 2θ=22.6, but rather were broad diffraction results. Therefore, it was understood that the crystal structure of cellulose was amorphized in samples treated under any atmosphere.

### Example 4

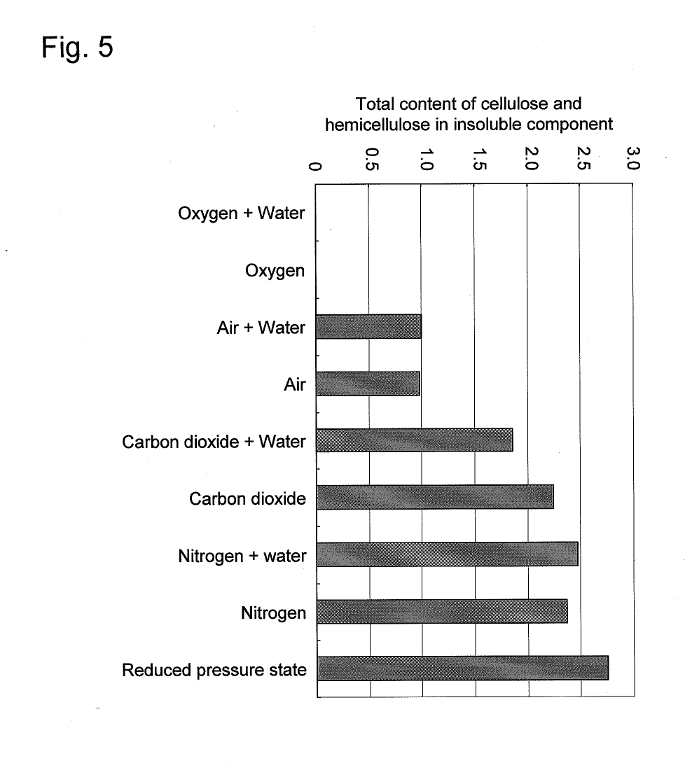

Each insoluble component separated in a manner similar to that in Example 1 was sufficiently washed with dimethyl sulfoxide (DMSO) and then further washed with a sufficient volume of distilled water. All the thus obtained residues were added to sulfuric acid. The sulfuric acid was subjected to component analysis, so that lower-molecular-weight components of cellulose and hemicellulose were quantitatively determined based on xylose content and glucose content. In addition, the total content of low-molecular-weight components (contained in insoluble components under a moisture-containing pseudo-air atmosphere) of cellulose and hemicellulose was designated as 1. Then, the aforementioned values were obtained as relative values with respect to the total content thereof. The graph in FIG. 5 shows the total contents of low-molecular-weight components of cellulose and hemicellulose contained in insoluble components at 24 hours after the initiation of treatment.

As shown in FIG. 5, in the case of results of treatment under a carbon dioxide, moisture-containing carbon dioxide, nitrogen, moisture-containing nitrogen, or reduced-pressure atmosphere, insoluble components contained higher amounts of low-molecular-weight components of cellulose and hemicellulose, compared with results of treatment under a pseudo-air or moisture-containing pseudo-air atmosphere. Specifically, it was understood that when a cellulosic biomass is treated with ionic liquid under a carbon dioxide, nitrogen, or reduced-pressure atmosphere, the rate of lowering the molecular weight for cellulose and hemicellulose is decreased, and also that low-molecular-weight components of cellulose and hemicellulose can be recovered as solid components.

Furthermore, insoluble components of samples treated under an oxygen or moisture-containing oxygen atmosphere contained no low-molecular-weight components of cellulose and hemicellulose. It was thus understood that the increased rate of lowering the molecular weight had caused the molecular weight of cellulose and hemicellulose contained in a cellulosic biomass to be lowered to a degree at which the same were soluble in ionic liquid.

The comprehensive conclusions of the results of Examples 1-4 are as follows. The rate of lowering the molecular weight of polysaccharides such as cellulose and hemicellulose contained in a cellulosic biomass can be controlled by changing the atmosphere under which ionic liquid is mixed with the cellulosic biomass. In particular, it was understood that treatment under an atmosphere with higher nitrogen partial pressure or carbon dioxide partial pressure than that of air or a reduced-pressure atmosphere results in a decreased rate of lowering molecular weight and makes it possible to maintain the solid state of low-molecular-weight components of cellulose and hemicellulose. Furthermore, conversely, it is better to mix ionic liquid with a cellulosic biomass under an atmosphere with higher oxygen partial pressure than that of air in order to increase the rate of lowering molecular weight.

It was also understood from the results of Example 3 that the rate of lowering molecular weight was decreased by the method for controlling the rate of lowering molecular weight according to the present invention, so that cellulose was amorphized in solid components (insoluble components) remaining unsolubilized. It was also understood from the results of Example 4 that in insoluble components, low-molecular-weight components of cellulose and hemicellulose are sufficiently present. In general, it is known that amorphization of a crystal structure effectively causes cellulose to be more susceptible to the effects of an enzyme. Solid components containing cellulose and hemicellulose obtained by decreasing the rate of lowering the molecular weight by the method for controlling the rate of lowering the molecular weight according to the present invention are thought to be more susceptible to the effects of an enzyme than untreated components. Therefore, it can be said that with regard to the method for controlling the rate of lowering the molecular weight according to the present invention, the step of decreasing the rate of lowering the molecular weight can be used as a pretreatment step for enzymatic hydrolysis (glycosylation).

1. A method for controlling the rate of lowering the molecular weight of polysaccharides contained in a cellulosic biomass, comprising mixing a cellulosic biomass with ionic liquid under an atmosphere with a partial pressure ratio differing from that of air.

2. The method for controlling the rate of lowering the molecular weight of polysaccharides contained in a cellulosic biomass according to claim 1, wherein the atmosphere has oxygen partial pressure higher than that of air.

3. The method for controlling the rate of lowering the molecular weight of polysaccharides contained in a cellulosic biomass according to claim 1, wherein the atmosphere has oxygen partial pressure lower than that of air.

4. The method for controlling the rate of lowering the molecular weight of polysaccharides contained in a cellulosic biomass according to claim 1, wherein the atmosphere has nitrogen partial pressure higher than that of air.

5. The method for controlling the rate of lowering the molecular weight of a cellulosic biomass according to claim 1, wherein the atmosphere has carbon dioxide partial pressure higher than that of air.

6. The method for controlling the rate of lowering the molecular weight of a cellulosic biomass according to claim 1, wherein the atmosphere is of a lower pressure than atmospheric pressure.

7. The method for controlling the rate of lowering the molecular weight of a cellulosic biomass according to claim 1, wherein the ionic liquid is a 1-ethyl-3-methylimidazolium salt.

8. The method for controlling the rate of lowering the molecular weight of a cellulosic biomass according to claim 7, wherein the 1-ethyl-3-methylimidazolium salt is 1-ethyl-3-methylimidazolium chloride.

9. A method for producing sugar, comprising:
a pretreatment step of mixing a cellulosic biomass with ionic liquid under an atmosphere with oxygen partial pressure lower than that of air;
a solid-liquid separation step of separating a solid component from a liquid component, both of which are obtained in the pretreatment step; and
a glycosylation step of glycosylating by enzyme treatment the solid component separated in the solid-liquid separation step.

10. The method for producing sugar according to claim 9, wherein the atmosphere further has nitrogen partial pressure higher than that of air.

11. A method for producing alcohol or organic acid, comprising:
a pretreatment step of mixing a cellulosic biomass with ionic liquid under an atmosphere with oxygen partial pressure lower than that of air;
a solid-liquid separation step of separating a solid component from a liquid component, both of which are obtained in the pretreatment step;
a glycosylation step of glycosylating by enzyme treatment the solid component separated in the solid-liquid separation step; and
a fermentation step of fermenting a sugar component obtained in the glycosylation step.

12. The method for producing alcohol or organic acid according to claim 11, wherein the atmosphere has nitrogen partial pressure higher than that of air.
